(19) United States
(12) Patent Application Publication
(10) Pub. No.: US 2023/0293077 A1
(43) Pub. Date: Sep. 21, 2023

(54) Patch Type Electrocardiogram Sensor

(71) Applicant: Byung Choo Moon, Toronto (CA)

(72) Inventor: Byung Choo Moon, Toronto (CA)

(21) Appl. No.: 18/319,847

(22) Filed: May 18, 2023

Related U.S. Application Data
(63) Continuation of application No. 17/682,345, filed on Feb. 28, 2022, now abandoned.

(51) Int. Cl. A61B 5/282; A61B 5/00; A61B 5/259; A61B 5/316

(52) U.S. Cl. CPC A61B 5/282; A61B 5/6823; A61B 5/259; A61B 5/316; A61B 2562/125; A61B 2562/166

(57) ABSTRACT

A patch type electrocardiogram (ECG) sensor including base layer having pad form; flexible printed circuit board (FPCB) layer formed on the base layer and having film form; sensor formed on FPCB layer and including plurality of electrodes configured to acquire ECG signals and plurality of electrode circuit parts which are constituted by circuit patterns and the first ends thereof are individually connected to the plurality of electrodes; main body formed on the FPCB layer and connected to second ends of the plurality of electrode circuit parts to converge the plurality of electrode circuit parts; and adhesive layer formed on FPCB layer such that plurality of electrodes are exposed and configured to attach the sensor to human body, connection part configured to transmit the ECG signals to the outside is installed at and connected to the main body, and main body and the connection part are connected by wire.

### BACKGROUND

### Field of the Invention

The present invention relates to a patch type electrocardiogram sensor which can be easily attached to body skin and measures a bio-signal.

### Discussion of Related Art

As an electrocardiogram (ECG), which is one type of typical bio-information, records action current generated when a myocardium contracts and relaxes due to a cardiac impulse, electrodes are attached to body skin, action current is measured according to myocardial contraction, and the data of the measured current is described in a graph.

Specifically, an active potential occurring when a myocardium contracts and relaxes due to a cardiac impulse generates a current transmitted from the heart to the whole body, the current generates potential differences according to positions of portions of the body, and the potential differences can be detected and recorded by electrodes attached to the body skin.

Such an ECG is used to check cardiac abnormalities and is used as a basic measurement method for diagnosing cardiac diseases such as angina, myocardial infarction, and arrhythmia.

Generally, an electrode induction method, which is used in clinics to measure an electrical anomaly of the heart by attaching two or more electrodes to the body skin, measures bio-potentials, which are generated when an electrical stimulation generated in a sinoatrial node of the heart is transmitted to left and right ventricles and the atria.

Electrodes of an ECG measurement apparatus can be divided into wet and dry electrodes according to whether a surface of the electrode contains an electrolyte.

When an electrode is a dry electrode, the electrode is typically attached to a chest region using a flexible band. In such a case, there is a problem in that it is difficult to wear the electrode for a long time because a feeling of tightness in the chest occurs when the electrode is attached to the chest.

Meanwhile, when an electrode is a wet electrode, the electrode is attached to a human body using an adhesive material and a separate band is not needed. However, a conventional wet electrode has problems in that pain accompanies the attachment or detachment of the electrode to or from the skin due to the adhesive material, it is difficult to closely contact with a human body part that is severely curved because the electrode is attached to the body using an adhesive pad, and thus, the quality of a cardiac impulse signal is lowered.

In addition, since a conventional ECG measurement apparatus is big and has many wires (electrical lines), there is a problem in that the use is cumbersome due to the wires becoming entangled and the like, and since a conventional ECG measurement sensor includes a protrusion-type knob formed at an attachment site thereof, there is a problem in that a patient feels uncomfortable when the sensor is attached to a back of the patient and the patient is lying on his/her back.

### SUMMARY OF THE INVENTION

The present invention is directed to providing a patch type electrocardiogram (ECG) sensor capable of being easily attached to a human body that is severely curved and including simplified wiring.

According to an aspect of the present invention, a patch type ECG sensor is provided including: a base layer having a pad form; a flexible printed circuit board (FPCB) layer formed on the base layer and having a film form; a sensor formed on the FPCB layer and including a plurality of electrodes configured to detect ECG signals and a plurality of electrode circuit parts which are constituted in circuit patterns and the end thereof are individually connected to the plurality of electrodes; a main body formed on the FPCB layer and connected to the other ends of the plurality of electrode circuit parts to converge the plurality of electrode circuit parts; and an adhesive layer formed on the FPCB layer such that the plurality of electrodes are exposed and configured to attach the sensor to a human body, wherein a connection part configured to transmit the ECG signals to the outside is installed at and connected to the main body, and the main body and the connection part are connected by a single wire.

### BRIEF DESCRIPTION OF THE DRAWINGS

The description above and other objects, features and advantages of the present invention will become more apparent to those of ordinary skill in the art by describing exemplary embodiments thereof in detail with reference to the accompanying drawings, in which:

FIG. 1 is a plan view of a structure of a patch type electrocardiogram (ECG) sensor according to one embodiment of the present invention;

FIG. 2 is a cross-sectional view illustrating the patch type ECG sensor shown in FIG. 1;

FIG. 3 is a view of the patch type ECG sensor, which is attached to a human body, according to one embodiment of the present invention; and

FIG. 4 is a block diagram illustrating a main body of the patch type ECG sensor according to one embodiment of the present invention.

### DETAILED DESCRIPTION OF EXEMPLARY EMBODIMENTS

The present invention relates to a patch type electrocardiogram (ECG) sensor which is easily attached to a human body, improves convenience during an operation by simplifying the wiring structure, and measures a bio-signal.

Here, an ECG represents a picture in which electrical activity of a heart is amplified and recorded, and such an ECG is used for measuring a heart condition or diagnosing the extent of heart damage.

The present invention relates to a patch type ECG sensor including: a base layer having a pad form; a flexible printed circuit board (FPCB) layer formed on the base layer and having a film form; sensors formed on the FPCB layer and including a plurality of electrodes configured to acquire ECG signals and a plurality of electrode circuit parts which are constituted by circuit patterns and the ends thereof are individually connected to the plurality of electrodes; a main body formed on the FPCB layer and connected to the other ends of the plurality of electrode circuit parts to converge the plurality of electrode circuit parts; and an adhesive layer formed on the FPCB layer such that the plurality of electrodes are exposed and configured to attach the sensors to a human body, wherein a connection part configured to transmit the ECG signals to the outside is installed at and connected to the main body, and the main body and the connection part are connected by a single wire.

Here, the main body may include: an amplifier configured to amplify the ECG signals to be transmitted through the plurality of electrode circuit parts; and a filter configured to filter the amplified ECG signals.

In addition, the adhesive layer may include a hydrogel component, and the electrode circuit parts may be formed by forming a conductive paste in circuit shapes on the adhesive layer through one method among silk screen, vacuum deposition, and sputtering deposition methods.

Embodiments of the present invention will be described in detail with reference to accompanying drawings. Before the embodiments are described, terms and words used in this specification and claims are not to be interpreted as limited to commonly used meanings or meanings in dictionaries and should be interpreted with meanings and concepts which are consistent with the technological scope of the invention based on the principle that the inventor has appropriately defined concepts of terms in order to describe the invention in the best way.

Therefore, since the embodiments described in this specification and configurations illustrated in the drawings are only exemplary embodiments and do not represent the overall technological scope of the invention, it should be understood that the invention covers various equivalents, modifications, and substitutions at the time of the filing of this application.

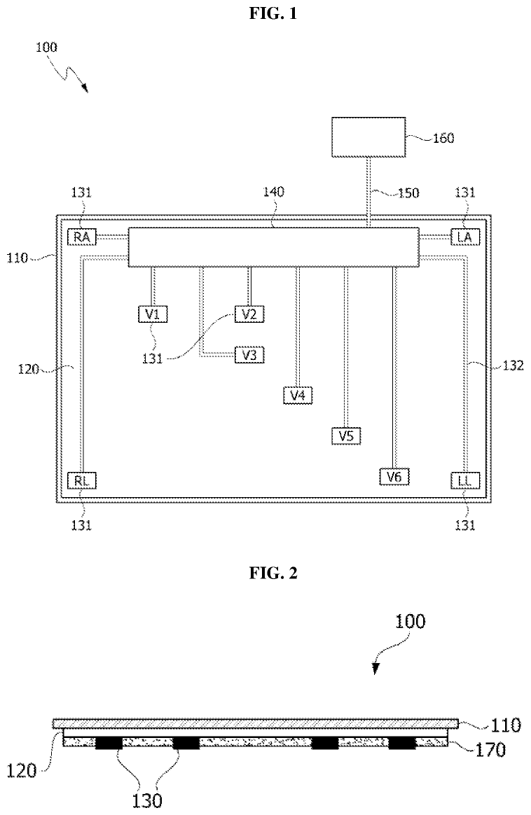

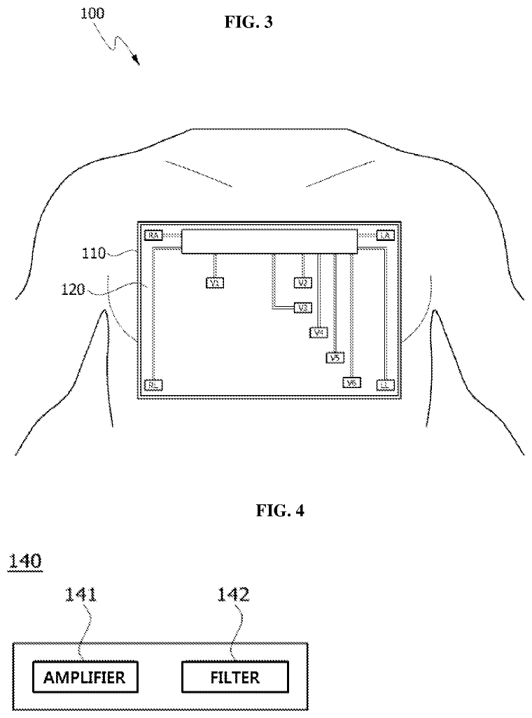

FIG. 1 is a plan view of a structure of a patch type ECG sensor according to one embodiment of the present invention, FIG. 2 is a cross-sectional view illustrating the patch type ECG sensor shown in FIG. 1, FIG. 3 is a view of the patch type ECG sensor according to one embodiment of the present invention, which is attached to a human body, and FIG. 4 is a block diagram illustrating a main body of the patch type ECG sensor according to one embodiment of the present invention.

Hereinafter, the patch type ECG sensor according to the present invention will be described in detail with reference to FIGS. 1 to 4 and the embodiments.

The present invention relates to a patch type ECG sensor 100 which is easily attached to a human body and improves convenience during an operation by simplifying wiring to measure a bio-signal.

Here, an ECG refers to a picture in which electrical activity of a heart is amplified and recorded, and such an ECG is used for measuring a heart condition or diagnosing the extent of heart damage.

In addition, wiring refers to a circuit configuration in which parts included in an apparatus are connected by electrical lines to form paths through which a current may flow.

As illustrated in FIGS. 1 and 2, the patch type ECG sensor 100 according to one embodiment of the present invention includes a base layer 110, a FPCB layer 120, an adhesive layer 170, sensors 130 having a plurality of electrodes 131 configured to acquire ECG signals and a plurality of electrode circuit parts 132 connected to the electrodes 131, and a main body 140 connected to the other ends of the plurality of electrode circuit parts 132 to converge the plurality of electrode circuit parts 132, which are formed on the FPCB layer 120.

In addition, a connection part 160 configured to transmit the ECG signals acquired by the main body 140 to the outside is formed installed at and connected to the main body 140, and here, the main body 140 and the connection part 160 are connected by a single wire 150. A more detailed explanation will be described below.

The base layer 110 according to one embodiment of the present may be formed in a pad form, made of a flexible synthetic resin film, and may form an external surface of the patch type ECG sensor 100.

In addition, the FPCB layer 120 may refer to a general FPCB and refer to an original board of a circuit board coated with a flexibly bent copper foil (copper film). Meanwhile, since the FPCB layer 120 according to one embodiment of the present invention is formed to suitably bend along a curved skin surface to which the FPCB layer 120 is attached, adhesion to the skin can be maximized.

In addition, the adhesive layer 170 according to one embodiment of the present invention may include a hydrogel component which is a conductive fluid capable of being in electrical contact with the body.

The adhesive layer 170 may include any transparent and viscous material, which has characteristics of maintaining adhesion even after being washed and reused, other than the hydrogel component and may be attached to or detached from the human body.

Referring to FIGS. 1 and 2, the FPCB layer 120 according to one embodiment of the present invention includes the sensors 130 and the main body 140.

First, the sensors 130 include the plurality of electrodes 131 configured to detect an action current of a heart and acquire ECG signals and the plurality of electrode circuit parts 132, constituted by circuit patterns, individually connected to the plurality of electrodes 131.

More specifically, the sensors 130 may acquire the ECG signals through the plurality of electrodes 131 configured to acquire the ECG signals from the human body. That is, the plurality of electrodes 131 included in the sensors 130 are attached to the skin of the human body, and potentials generated at the skin by action currents generated from a myocardium may be detected through the plurality of electrodes. Accordingly, an ECG signal acquired from a sensor 130 may be in the form of potential difference.

For reference, although a magnitude of the ECG signal may be different according to the position with which an electrode 131 is in contact, a shape of the ECG signal is the same and a time delay of the ECG signal is very small.

Snap electrodes made of a conductive metal may be used for and applied to such electrodes 131, and in addition, the electrodes 131 may also be made of a biocompatible material.

Here, the biocompatible material electrode obtained by stirring and curing a biocompatible silicone with a conductive material (carbon nanotube (CNT)), is utilized to induce bio-potential by being in direct contact with the skin. The biocompatible material electrode may have a thickness of several millimeters.

In addition, a biocompatible material, which is a polymeric material which does not adversely affect human body tissue or a biological material even when being in contact therewith for a long period of time, may be used as equipment in a medical field including dialysis films for artificial kidneys, artificial blood vessels, artificial teeth, blood storage bags for blood transfusion, and tubes used for blood circuits, and as the material in a medical field including polyvinyl chloride, silicone, Teflon, or the like.

Particularly, the electrode circuit parts 132 according to one embodiment of the present invention are used to connect the plurality of electrodes 131 and the main body 140 which will be described below, and are insulated except for both ends thereof. The electrode circuit parts 132 may be made by forming patterns with a biocompatible material, and also be used by forming a conventionally used circuit which is short.

In addition, the electrode circuit parts 132 may be formed by forming a conductive paste in circuit shapes on the FPCB layer 120 through one method among silk screen, vacuum deposition, and sputtering deposition methods.

Next, the main body 140 according to one embodiment of the present invention is provided so as to converge the plurality of electrode circuit parts 132, and the connection part 160, which will be described below, and the main body 140 may be connected by the single wire 150.

That is, since the plurality of electrode circuit parts 132 are converged and integrated into the single wire 150 from the main body 140 according to one embodiment of the present invention, there is an effect in which convenience during operation is improved.

Meanwhile, in the patch type ECG sensor 100 according to one embodiment of the present invention, the plurality of electrodes 131 may be formed on the FPCB layer 120 by referencing the chest lead electrodes.

As illustrated in FIG. 1, ten electrodes 131 may be formed on the FPCB layer 120 according to one embodiment of the present invention. More specifically, six electrodes (V1 to V6), which are chest lead electrodes, among the ten electrodes may be formed to be positioned according to a unipolar chest lead method. More specifically, the electrode V1 positioned at a fourth intercostal space to the right of the sternum of a patient, the electrode V2 positioned at the fourth intercostal space to the left of the sternum of a patient, the electrode V3 positioned midway between the electrodes V2 and V4, and the electrode V4 positioned on a mid-clavicular line at a height of a fifth intercostal space may be formed on the FPCB layer 120 according to one embodiment of the present invention. In addition, the electrode V5 may be positioned on an anterior axillary line (the front side of the armpit) at the same level as that of the electrode V4, and the electrode V6 may be positioned on a mid-axillary line (the middle of the armpit) at the same level as that of the electrode V4 may be formed on the FPCB layer 120 according to one embodiment of the present invention.

In addition, the remaining four electrodes RA, RL, LA, and LL) may be formed to be attached to the left and right upper portions and left and right lower portions of the chest of the patient.

FIG. 3 is a view of the patch type ECG sensor 100 according to one embodiment of the present invention, which is attached to a human body.

Referring to FIG. 3, an ECG may be measured through the chest lead method using the patch type ECG sensor 100 according to one embodiment of the present invention.

That is, it takes a long time to individually attach the plurality of electrodes 131 to a patient, and it is difficult to find accurate attachment positions. However, in the patch type ECG sensor 100 according to one embodiment of the present invention, since the FPCB layer 120 includes the plurality of electrodes 131 thereon, the ECG signals may be measured quickly and accurately even when making a response to an emergency situation.

However, the positions of the above-described electrodes 131 are assigned according to one embodiment and are not limited thereto.

In addition, referring to FIG. 4, the main body 140 according to one embodiment of the present invention includes an amplifier 141 configured to amplify ECG signals acquired through the electrodes 131 and a filter 142 configured to remove noise signals from the amplified ECG signals.

Particularly, the ECG signals (current signals) measured by the electrodes 131 are transmitted to the main body 140 through the electrode circuit parts 132, the transmitted current signals are amplified by the amplifier 141 in the main body 140, filtered by the filter 142, and transmitted to the connection part 160 which will be described below.

More specifically, the amplifier 141 according to one embodiment of the present invention amplifies the ECG signals acquired from the electrodes 131 and may combine and amplify the ECG signals in the form of potential difference detected by the plurality of electrodes 131.

In addition, the filter 142 according to one embodiment of the present invention is for removing the noise signals from the amplified ECG signals and may remove the noise signals from the ECG signals caused by various bio-currents.

Meanwhile, in the patch type ECG sensor 100 according to one embodiment of the present invention, the connection part 160 may be installed and connected to the main body 140. Here, the ECG signals processed by the amplifier 141 and the filter 142 in the main body 140 are transmitted to the connection part 160 through the single wire 150.

Here, the connection part 160 is a type of connecter capable of being in contact with and being connected to an external device, and the connecter may transmit the ECG signals to a computer, a medical device, or a smart device (a smart phone, a smart pad, etc.) through a wired method, and thus the ECG signals may be monitored.

However, the connection part 160 is not limited thereto. As another aspect, a wireless communication device may be connected to the connection part 160 to transmit ECG signals to an external device through a wireless communication method such as Bluetooth, a wireless local area network (WLAN), or ZigBee.

As described above, a patch type ECG sensor according to one embodiment of the present invention is miniaturized by forming electrodes configured to detect action currents of a heart and circuit patterns on a FPCB layer and has an effect in which convenience during operation is improved by integrating a plurality of electrode circuit parts and the like into a single wire from a main body.

In addition, since a plurality of electrodes according to the embodiment of the present invention are formed on a single patch, various waveforms of ECG can be simultaneously measured, and even an inexperienced user with respect to ECG measurement can simply attach the electrodes to accurate positions of a patient.

In addition, a patch type ECG sensor according to the present invention can be formed in a patch type and attached to a chest or back region to measure ECG.

1. A patch type electrocardiogram (ECG) sensor comprising:
a base layer having a pad form;
a flexible printed circuit board (FPCB) layer formed on the base layer and having a film form;
a sensor formed on the FPCB layer and including a plurality of electrodes configured to acquire ECG signals and a plurality of electrode circuit parts which are constituted by circuit patterns and the first ends thereof are individually connected to the plurality of electrodes;
a main body formed on the FPCB layer and connected to the second ends of the plurality of electrode circuit parts to converge the plurality of electrode circuit parts; and
an adhesive layer formed on the FPCB layer such that the plurality of electrodes are exposed and configured to attach the sensor to a human body,
wherein a connection part configured to transmit the ECG signals to the outside is installed at and connected to the main body, and the main body and the connection part are connected by a single wire.

2. The patch type ECG sensor of claim 1, wherein the main body includes:
an amplifier configured to amplify the ECG signals to be transmitted through the plurality of electrode circuit parts; and
a filter configured to filter the amplified ECG signals.

3. The patch type ECG sensor of claim 1, wherein the adhesive layer includes a hydrogel component.

4. The patch type ECG sensor of claim 1, wherein the electrode circuit parts are formed by forming a conductive paste in circuit shapes on the adhesive layer through one method among silk screen, vacuum deposition, and sputtering deposition methods.
